(19) United States
(12) Patent Application Publication
(10) Pub. No.: US 2005/0113673 A1
(43) Pub. Date: May 26, 2005

(54) Method and system to reduce motion-related image artifacts during breath holding

(76) Inventors: Gobal B. Avinash, New Berlin, WI (US); Prathyusha K. Salla, Waukesha, WI (US)

Correspondence Address: Patrick S. Yoder;FLETCHER YODER, P.O. Box 692289, Houston, TX, 77269-2289, US

(21) Appl. No.: 10/723,859

(22) Filed: Nov. 26, 2003

(51) Int. Cl. A61B 5/04; A61B 5/05

(52) U.S. Cl. 600/413; 600/428; 600/509

(57) ABSTRACT

One or more techniques are provided for gating the acquisition or selection of image data based upon the respiration of a patient. The technique provides for the acquisition of respiratory motion data from the image system or from one or more sensor-based motion determination systems. Various attributes of respiratory motion may be derived from the respiratory motion data and motion thresholds determined from the attributes. Based upon the respiratory motion of the patient and the determined thresholds, image data may be acquired or selected which corresponds to one or more breath-holds by the patient or low respiratory motion quiet periods within the breath-holds. The technique may be implemented in a fully automated or operator assisted or supervised manner.

### BACKGROUND OF THE INVENTION

The present technique relates generally to the correction and/or prevention of motion-related artifacts in medical imaging. More specifically, the present technique relates to the use of a respiration sensor during medical imaging to facilitate image acquisition or selection during intervals of breath holding.

In the medical field, it is often desirable to generate images of the internal organs or structure of a patient for diagnosis or examination. For example, magnetic resonance imaging and computed tomography are two well known examples of imaging modalities used to generate images of the internal organs or structures of a patient. The reconstructed images, however, may be flawed or contain artifacts due to the motion of internal organs, such as the heart, lungs, diaphragm, stomach, and so forth. In particular, if the imaged region has undergone motion during the imaging process, various motion-related artifacts or discontinuities may be present in the reconstructed image.

For example, images acquired of one or more organs in the torso of a patient, such as the heart, lungs, stomach, and so forth, may have motion-related artifacts associated with cardiac and/or respiratory activity. One technique that may be employed to minimize or prevent artifacts related to respiration is respiration gating, i.e., acquiring or selecting image data associated with low-motion phases of the respiratory cycle. The effectiveness of respiration gating may be enhanced or prolonged by requesting that the patient hold her breath during image acquisition, thereby providing an extended period of little or no respiratory motion. After a certain interval or after visual confirmation of breath-holding, an operator may acquire the desired image data or may note the start and stop times of breath-holding to allow selective processing of the acquired image data.

However, images acquired using respiration gating and breath-holding techniques may still exhibit some motion-related artifacts. For example, to the extent an operator is involved, the operator may fail to properly note the desired imaging interval associated with the breath-hold by either over or under-estimating the interval. If the interval is over-estimated, motion artifacts may be exacerbated due to the extreme inhalation and exhalation motions associated with holding one's breath. If the interval is under-estimated, useful image data may be missed, potentially impacting image quality and the diagnostic value of the images. Furthermore, other motions, including body movement, may also affect the image quality without being noted or detected by the operator. Therefore, it may be desirable to determine the existence and duration of a breath-hold more accurately during image acquisition.

### BRIEF DESCRIPTION OF THE INVENTION

The present invention is directed to a technique for detecting and/or measuring the duration of a breath-hold during image acquisition. The present technique provides for the measurement of the motion of the chest wall during image acquisition, using sensor or image-based techniques. The motion may be analyzed in real time and used to start and stop acquisition, either automatically or via notification of the operator. The decision to start and stop acquisition may be based on a metric derived from the analysis. Alternatively, the motion may be analyzed retrospectively and used to selectively process a continuous or extended image data set.

In accordance with one aspect of the present technique, a method for gating image data is provided. In the present technique, a set of motion data is acquired during a breath hold. One or more attributes of motion are derived from the set of motion data. An initiation threshold and a termination threshold are derived from the one or more attributes. A set of gated image data may be generated using one or more gating intervals derived from the initiation threshold and the termination threshold. Systems and computer programs that afford functionality of the type defined by this method are also provided by the present technique.

### BRIEF DESCRIPTION OF THE DRAWINGS

The foregoing and other advantages and features of the invention will become apparent upon reading the following detailed description and upon reference to the drawings in which:

FIG. 1 is a general diagrammatical representation of certain functional components of an exemplary generic imaging system capable of gating via the present technique;

FIG. 2 is a flowchart depicting the acquisition of data using respiration gating, in accordance with the present technique;

FIG. 3 is a flowchart depicting the selection of acquired data using respiration gating, in accordance with the present technique;

FIG. 4 is a flowchart depicting a manual implementation of respiration gating, in accordance with the present technique; and

FIG. 5 is a flowchart depicting the acquisition of data using respiration gating and additional scan parameters, in accordance with the present technique.

### DETAILED DESCRIPTION OF SPECIFIC EMBODIMENTS

In the field of medical imaging, the motion of an organ may lead to motion artifacts in images of the organ. Various techniques may be employed to address the motion of the imaged organ. For example, gating techniques may be employed which selectively acquire or process image data in accordance with what is known of the motion of the organ of interest. In general, gating techniques that allow selective acquisition of image data are known as prospective gating techniques. Conversely, gating techniques that allow selective processing of an already acquired image data set are known as retrospective gating techniques. Combination or composite gating techniques, such as those involving both prospective and retrospective gating or those incorporating motion compensation, may also be employed.

For example, in instances where respiratory motion may produce motion-related artifacts in images, respiration gating may be employed to acquire image data when pulmonary motion is minimal, such as subsequent to an exhalation but prior to an inhalation. Alternatively, respiration gating may be employed to selectively process an image data set which has already been acquired, such as by processing only that image data corresponding to the desired respiratory phase. To improve the effectiveness of respiration gating, the patient may be asked to hold her breath, creating a longer interval of reduced pulmonary motion and, therefore, a longer potential gating interval.

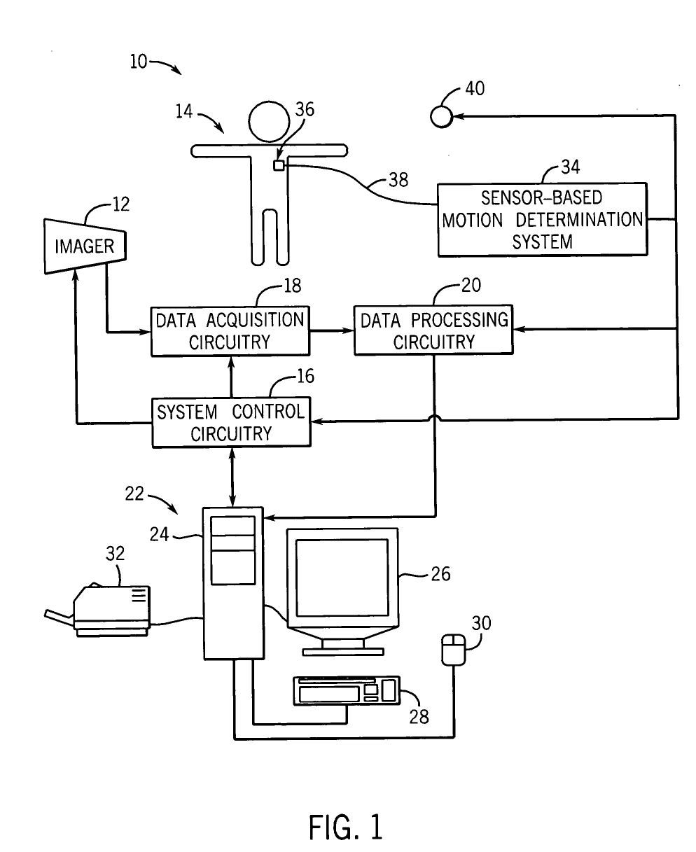

An exemplary imaging system 10 capable of operating in accordance with the present technique is depicted in FIG. 1. Generally, the imaging system 10 includes some type of imager 12 that detects signals and converts the signals to useful data. As described more fully below, the imager 12 may operate in accordance with various physical principals for creating the image data. In general, however, the imager 12 creates image data indicative of the region of interest in a patient 14, either in a conventional support, such as photographic film, or in a digital medium.

The imager 12 operates under the control of system control circuitry 16. The system control circuitry 16 may include a wide range of circuits, such as radiation source control circuits, timing circuits, circuits for coordinating data acquisition in conjunction with patient or table movements, circuits for controlling the position of radiation sources and detectors, and so forth. In the present context, the system control circuitry 16 may also include memory elements, such as magnetic or optical storage media, for storing programs and routines executed by the system control circuitry 16 or by associated components of the system 10. The stored programs or routines may include programs or routines for performing all or part of the present technique.

Image data or signals acquired by the imager 12 may be processed by the imager 12, such as for conversion to digital values, and provided to data acquisition circuitry 18. The data acquisition circuitry 18 may perform a wide range of processing functions, such as adjustment of digital dynamic ranges, smoothing or sharpening of data, as well as compiling of data streams and files, where desired. In situations where pre-acquisition image data, such as Navigator pulses in magnetic resonance imaging (MRI), are acquired, the data acquisition circuitry 18 may provide image data to the system control circuitry 16 for prospective gating.

The data acquisition circuitry 18 may also transfer acquisition image data to data processing circuitry 20, where additional processing and analysis are performed. The data processing circuitry 20 may perform substantial analyses of image data, including ordering, sharpening, smoothing, feature recognition, and so forth. In addition, the data processing circuitry 20 may receive motion data for one or more organs from one or more sensor-based motion detection systems 34, as discussed in detail below. Based on image-based and/or sensor-based motion data, respiration gating may be facilitated by the data processing circuitry 20, such as by determining motion attributes, motion thresholds, and/or gating intervals that may be provided to the system control circuitry 16 and/or operator workstation 22. The processed image data may be stored in short or long term storage devices, such as picture archiving communication systems, which may be located within or remote from the imaging system 10 and/or reconstructed and displayed for an operator, such as at the operator workstation 22.

In addition to displaying the reconstructed image, the operator workstation 22 may control the above-described operations and functions of the imaging system 10, typically via an interface with the system control circuitry 16. The operator workstation 22 may include one or more processor-based components, such as general purpose or application specific computers 24. In addition to the processor-based components, the operator workstation 22 may include various memory and/or storage components including magnetic and optical mass storage devices, internal memory, such as RAM chips. The memory and/or storage components may be used for storing programs and routines for performing the techniques described herein that are executed by the operator workstation 22 or by associated components of the system 10. Alternatively, the programs and routines may be stored on a computer accessible storage and/or memory remote from the operator workstation 22 but accessible by network and/or communication interfaces present on the operator workstation 22.

The operator workstation may also comprise various input/output (I/O) interfaces, as well as various network or communication interfaces. The various I/O interfaces may allow communication with user interface devices, such as a display 26, keyboard 28, mouse 30, and printer 32, that may be used for viewing and inputting configuration information and/or for operating the imaging system 10. The various network and communication interfaces may allow connection to both local and wide area intranets and storage networks as well as the Internet. Though the various I/O and communication interfaces are indicated as operating through wires or lines in FIG. 1, it is to be understood that wireless interfaces may also be utilized where appropriate.

As one of ordinary skill in the art will appreciate, more than a single operator workstation 22 may be provided for an imaging system 10. For example, an imaging scanner or station may include an operator workstation 22 which permits regulation of the parameters involved in the image data acquisition procedure, whereas a different operator workstation 22 may be provided for manipulating, enhancing, and viewing results and reconstructed images.

The motion of the lungs or other respiratory organs of interest, such as the diaphragm, may be measured in a variety of ways. As one of ordinary skill in the art will readily apprehend, the type of data gating desired, i.e., prospective or retrospective, may determine the type of motion data acquired. In some cases, the motion data of interest may be derived using the image scanner 12 itself. For example, pre-acquisition imaging techniques, such as navigator pulses in MR systems, scout images in CT systems or fluoroscopic images in other generalized X-ray applications, may be employed to determine the motion of the lungs, diaphragm, chest wall, and so forth, as indicators of respiration. Pre-acquisition motion detection and measurement typically involves determining the position of the organ or organs of interest by a pre-acquisition measurement using the imaging system 10. Subsequent image acquisition can then occur during similar states of organ motion or subsequently acquired image data may be selected for processing and reconstruction based upon a similar state of organ motion.

For example, in MRI, a navigator echo pulse is a quick MR pre-pulse sequence that measures the position of an organ, such as the diaphragm, before primary image data acquisition. The pre-pulse sequence images a narrow area perpendicular to the movement of the organ of interest, i.e., a vertical area for a diaphragm. The contrast of the moving interface is typically high to permit easy automatic detection. Once the pre-acquisition motion data has been acquired, the position of the interface may be recorded and imaging data may be acquired or selected based on whether the position of the interface falls within a range of pre-specified values determined from the pre-acquisition data. Using the navigator echo data, similar respiratory motion or other motion states of the patient can be identified and used for motion estimation. Hence, the navigator echo technique may be used as a respiratory gating technique that does not utilize additional sensing equipment, as the MR system itself provides the sensing.

Similarly, motion data derived from the acquired images, such as from the acquired and/or reconstructed image domains, may be used to determine the motion of the one or more respiratory organs. The motion data may be determined from one-dimensional, two-dimensional, or three-dimensional representations of the imaged region that are derived from the image data or from the raw image data itself. For example, organ motion may be detected and/or measured in the acquired or reconstructed image domain after a segmentation or structure identification step. In particular, a segmentation step may identify the chest wall or a boundary region of the lungs or diaphragm in the acquired or reconstructed image domains. Changes in the location of the wall or boundary region in the sequential image date may then be used to detect and/or measure respiratory motion in the patient. Uses of the imaging system 10 to acquire motion data, either in the pre-acquisition or in the post-acquisition context, are examples of image-based motion determination, as discussed in detail herein.

Alternatively, sensor-based motion determination techniques may be employed in conjunction with or instead of image-based techniques. In these instances, the exemplary imaging system 10 may include or may be in communication with one or more sensor-based motion determination systems 34. The sensor-based motion determination systems 34 typically comprise one or more sensors 36 in the form of a pad or contact that may be disposed on skin surface of the patient 14. The contact area of a sensor 36 may vary in size from micrometers to centimeters in diameter and height. The size selected is usually based on an application. Similarly, the number of sensors 36 used may depend on the application.

When disposed on the patient 14, the sensor 36 may detect and/or measure some metric or parameter of interest, such as an electrical or mechanical event, that may be used as an indicator of respiratory motion. The sensor 36 may be connected to the respective sensor-based determination system 34 via one or more leads 38 which may transmit a signal representative of the sensed metric or parameter to the respective system 34 for processing. In some contexts, the sensor 36 may communicate with the respective sensor-based motion detection system 34 via wireless means, such as a wireless network protocol, as opposed to a physical lead 38.

Sensor-based systems 34 may measure electrical activity or characteristics of a respiratory organ to determine motion. For example, electrical activity indicative of the muscular contractions of an organ may be measured. Alternatively, changes in electrical properties that are indicative of organ motion may be measured, such as in impedance plethysmography. The sensors 36 used to detect electrical events, such as electrical contact pads, are typically strategically placed to detect the electrical attributes of the organ.

Sensor-based motion determination measurement systems 34 may instead measure non-electrical activity or characteristics to determine respiratory motion. For example, internal movement caused by respiration may create mechanical motion detectable by one or more suitable sensors 36 disposed on the skin of the patient 14 as pressure, displacement, acceleration, velocity, pressure, and/or other mechanical indicators of motion. In this manner, internal motion of one or more respiratory organs may be detected and/or measured by various types sensors 36, including accelerometers, optical markers, displacement sensors, force sensors, ultrasonic sensors, strain gauges, photodiodes, and pressure sensors.

Whether measuring electrical or non-electrical activity, one or more sensors 36 may be employed. The sensors 36 may be arranged in an array or matrix format placed in or near the region of interest. Sensor arrays or configurations are possible in which the sensors 36 are arranged in a three-dimensional matrix such that the entire body surface in the region of interest is covered, such as by using a suit or wrap. Typically, in an array of sensors 36 used to measure non-electrical events, the sensors 36 are placed equidistant from each other. For instance, a δ unit of separation may be maintained between the sensors 36 in the X, Y, and Z directions.

While the motion information, whether determined by image-based or sensor-based means, is useful for respiration gating, it may also be used to provide feedback to the patient 14 or an operator regarding the patient's breath hold status. For example, a feedback device 40, such as a visual indicator or audio indicator, may provide motion information to the patient 14 from the sensor-based motion determination system 34, the data processing circuitry 20, and/or the system control circuitry 16. An indication that the level of patient motion, particularly respiratory motion, is acceptable for imaging may be provided to the patient in the form of a colored light, displayed text or symbol, or audible tone or message. Similarly, an indication that the level of patient motion is unacceptable for imaging may be provided to the patient 14 in similar manners. For example, a green light might be lit to indicate acceptable breath-holding motion and a red light to indicate unacceptable breath-holding motion. The acceptable or unacceptable indications may be determined using the techniques described below, i.e., derived motion attributes and thresholds, or by comparison of the motion data to arbitrary criteria, such as an operator or pre-configured motion threshold.

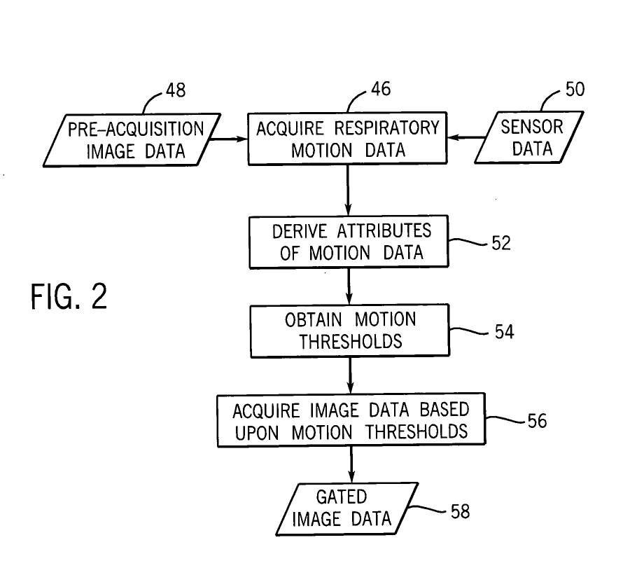

The exemplary imaging system of FIG. 1 may image one or more organs affected by respiratory motion using image-based and/or sensor-based motion determinations to facilitate respiration gating. For example, prospective respiration gating may be performed using the system of FIG. 1, with or without operator assistance, as depicted in FIG. 2. In the prospective gating example, respiratory motion data may be acquired, as depicted at step 46, from a set of pre-acquisition image data 48 and/or from one or more sets of sensor data 50.

As noted above, the pre-acquisition image data 48 may include Navigator pulses in an MRI system, scout images in a CT system, or fluoroscopic images in a digital X-ray based system. The sensor data 50 may include measures of electrical and/or non-electrical activity or indicators of respiratory motion. For example, the sensor data 50 may include the data obtained by a single displacement sensor disposed on the chest of the patient 14 to measure the displacement of the chest wall during respiration. In the absence of respiration, i.e., during the breath-hold, the displacement sensor may also be used to measure other body movements for consideration in the gating process or during evaluation of data quality. The sensor data may also include the data obtained by an array of electrodes disposed on the chest wall to provide impedance plethysmography data.

The acquisition of motion data depicted at step 46 may begin prior to when the patient commences holding his breath. For example, the breathing pattern of the patient 14 may monitored for several respiratory cycles, such as 5 to 10 cycles, prior to a breath-hold. The acquired motion data may be processed to derive various motion attributes, as depicted at step 52. For example, motion attributes such as the periodicity of the respiratory cycles and/or the range of the measured parameter, such as chest wall motion or impedance, may be determined. Similarly, a running average of temporal differences may be determined. These various attributes of the motion data may provide a set of baseline conditions that may be used in evaluating the respiration of the patient 14 to determine the initiation and termination of breath-holds.

The various attributes determined at step 52 may be used to obtain motion thresholds, as depicted at step 54. The motion thresholds, which may be based on temporal differences, displacement, periodicity, impedance, and so forth, may be compared to current motion data to determine the onset and end of breath-holds or of a quiet period corresponding to the low respiratory motion interval within the breath-hold. The threshold ranges may be selected based upon a breathing pattern analysis of the respiration of the patient 14 over a desired time interval, such as a 5 to 30 second interval. Alternatively, the operator may manually input or select the threshold for the patient 14, such as after visually reviewing the respiratory motion data at the operator workstation 22.

For example, assuming temporal difference is measured as an indicator of respiratory motion, at the beginning of a breath-hold the temporal differences will typically be higher than the running average of the temporal differences as the patient 14 heaves for a breath. Subsequently, the temporal differences will decrease below a threshold value, obtained from the motion attributes and/or the baseline conditions, indicating the initiation of the breath-hold. Similarly, the current temporal differences decreasing below the threshold, or some determined time interval subsequent to this event, may correspond to the onset of the quiet period within the breath-hold.

Acquisition of the image data may be started when the breath-hold or quiet period has been initiated, as determined by the measured data and determined initiation threshold, as depicted at step 56. Similarly, the acquisition may be terminated when the temporal difference, or other parameter of interest, exceeds a threshold associated with the end of the quiet period or breath hold. Typically, the termination threshold will be larger by some factor than the changes tolerated in the parameter during acquisition. The tolerable range of motion for the parameter during image acquisition may be determined from data acquired from the current patient, such as during patient preparation or pre-acquisition, or from multiple patients, such as a historical population. The result of the initiation and termination of the image data acquisition process based upon the measured motion data and determined motion thresholds, as depicted at step 56, is a set of gated image data 58. The gated image data 58 represent image data acquired during one or more breath-holds or the quiet periods associated with those breath-holds. The gated image data 58 may be reconstructed to generate medically useful images with a reduced incidence of motion artifacts related to respiration.

In addition, statistical analysis of the acquired motion data during image acquisition may be performed as an external metric for measuring the quality of the acquired image data. As a result, data obtained during relatively noisy or restless breath-holds may be discarded automatically or at the discretion of the operator. Similarly, non-respiratory motions of the patient that may be noted in the acquired motion data, such as by one or more displacement sensors, may result in the automatic or operator-assisted discard of image data obtained during a quiet period or a breath hold that is unacceptable due to patient motion.

Though the preceding example discusses the use of temporal difference as a metric, other parameters, as noted above, may be employed in addition to or instead of temporal difference. For example, the displacement or absolute motion of the chest wall may be measured, and suitable thresholds determined, from the acquired motion data. Similarly, chest wall location, velocity, pressure, and/or acceleration may provide comparable ranges and possible thresholds. In addition, impedance or other electrical characteristics may be measured and used to ascertain thresholds indicative of the onset and termination of a breath hold or the quiet period associated with the breath-hold.

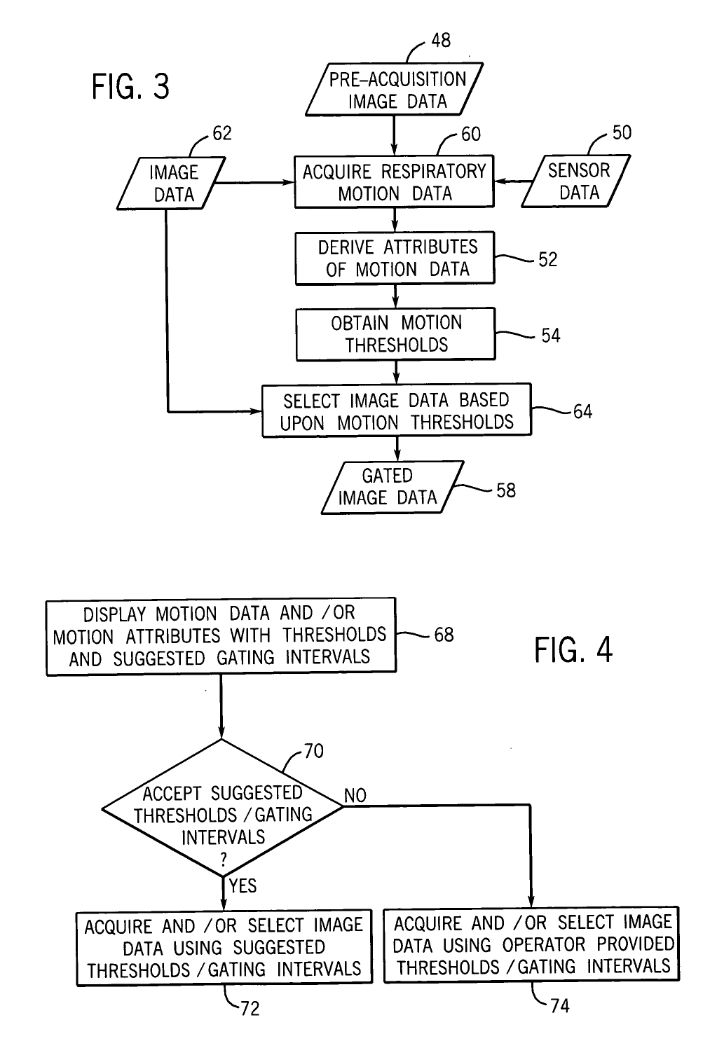

Alternatively, retrospective respiration gating may be performed using the respiratory motion data, as depicted in FIG. 3. In the retrospective gating example, respiratory motion data may be acquired, as depicted at step 60, from a set of image data 62, pre-acquisition image data 48, and/or from one or more sets of sensor data 50. As previously discussed, the sensor data 50 may include measures of electrical and/or non-electrical activity or indicators of respiratory motion. Similarly, the pre-acquisition image data 48, depending on the imaging modality, may include Navigator pulses, scout images, fluoroscopic images, and so forth, as previously discussed. The image data 62, however, may consist of a full or partial set of image data acquired during the execution of a standard imaging protocol of the imaging modality. The acquisition of the respiratory motion data at step 60 may occur in the acquisition or reconstruction domains of the image data 62. In particular, image data 62 acquired from the acquisition or reconstruction domains may be processed to segment or identify structures of interest, which may then be sequentially analyzed to acquire motion data for one or more respiratory structures at step 60. For example, the chest wall, pulmonary edges, diaphragm edges, and so forth may be segmented and located in successive image data to provide respiratory motion data.

The motion data acquired at step 60 may represent motion data acquire prior to the initiation of breath-holding. For example, the breathing pattern of the patient 14 may be determined from motion data pertaining to a number of respiratory cycles, typically 5 to 10 cycles, preceding breath-holding. As previously discussed, the acquired motion data may be processed to derive various motion attributes, as depicted at step 52, which may be used to obtain the desired motion thresholds at step 54, as discussed in the context of prospective gating.

However, in the retrospective gating process, the motion attributes and thresholds are not used to activate and deactivate the imager 12 or data acquisition circuitry 18. Instead, the motion attributes and thresholds are used to select a set of gated image data 58 from the image data 62, as depicted at step 64. As previously discussed, the gated image data 58 corresponds to image data 62 acquired during one or more breath-holds and/or quiet periods associated with such breath-holds. In this manner the image data 62 may be selectively processed such that the resulting images are generated using image data acquired during the breath-holds or quiet periods within the breath-holds. In addition, the motion data may be used, as discussed above, to provide an external metric of data quality measure and/or to discard unacceptable image data acquired during a breath-hold.

The respiration gating techniques discussed herein may be used to automatically acquire and/or select image data, as depicted at steps 56 and 64 of FIGS. 2 and 3, respectively. In particular, automated routines or programs running on suitable components of an imaging system 10 may perform the described functions. For example, the acquisition of respiratory motion data, derivation of motion attributes and suitable thresholds, and acquisition and/or selection of image data may be implemented automatically by components of the imaging system 10 by respective routines. In this manner, the start and stop of image data acquisition via prospective respiration gating may be automated after an operator initiates the scan protocol. Alternatively, in an automated retrospective implementation, the selection of image data for processing or reconstruction may be automated.

The present respiration gating techniques may also be implemented with some degree of operator input, as depicted in FIG. 4. For example, the acquisition step 56 and/or selection step 64 may include displaying the motion data and/or motion attributes in conjunction with the suggested thresholds and/or gating intervals, as depicted at step 68. The information displayed in this manner may be displayed at the operator workstation 22. The operator may then decide whether to accept or reject the suggested thresholds and/or gating intervals, as depicted at decision block 70. If accepted, the image data may be acquired or selected based upon the suggested thresholds and/or gating intervals, as depicted at step 72. If, however, the operator is not satisfied with the suggested thresholds and/or gating intervals, the operator may provide the desired thresholds and/or gating intervals, as depicted at step 74. In this manner, some degree of operator control may be retained where desired to fine tune or customize the imaging and respiration gating process for problematic patients.

Furthermore, the present respiration gating techniques may facilitate imaging based upon operator selected scan parameters, which might otherwise require substantial operator oversight or involvement. For example, an operator may specify that the imaging protocol comprise a designated number of slices or images, a designated duration, or other imaging protocol criteria. For example, a typical MR protocol may specify the acquisition of ten slices during the breath-hold or quiet period, each slice requiring ten seconds to acquire. Similarly, a CT protocol may specify that a certain number of images be acquired during the breath-hold or quiet period and an X-ray protocol may specify a desired exposure duration during the breath-hold or quiet period.

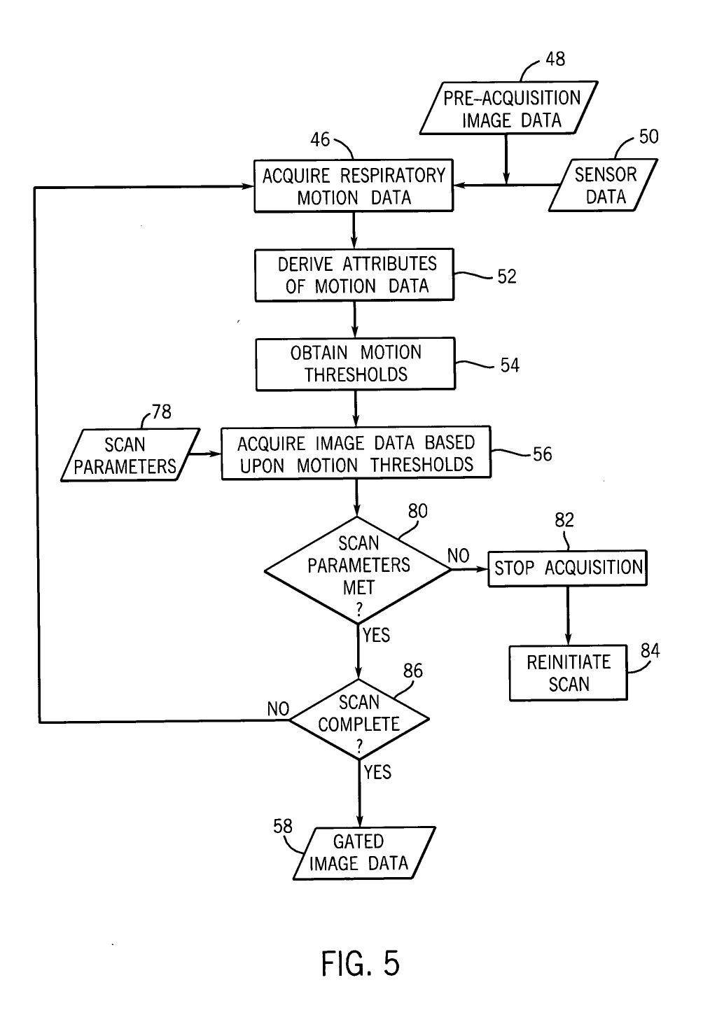

The present technique may facilitate satisfying such criteria, as depicted in FIG. 5. In the depicted example, the operator may specify, by selecting a protocol or by arbitrarily designation, one or more scan parameters 78. As noted above, these scan parameters 78 may include the number of slices or images to be acquired during the breath hold or quiet period and/or an exposure duration. In a prospective gating context, the image data acquisition may proceed, as discussed above with regard to FIG. 2. For each specified exposure, slice, and/or image, a determination may then be made, at decision block 80, whether the specified scan parameter was fulfilled in view of the determined breath hold or quiet period interval. If the scan parameter 78 was not satisfied, acquisition may be stopped and the operator notified, as depicted at step 82. The operator may then reinitiate the scan at step 84. If the scan parameter 78 is satisfied, acquisition continues until the scan is complete, as determined at decision block 86, and a set of gated image data 58 is generated.

For example, if acquisition of ten MR slices has been specified, each slice to be acquired during a ten second quiet period, the determination at decision block 80 may determine whether the acquisition quiet period was sufficient to meet the input scan parameter 78. If the quiet period for each slice is sufficient, acquisition proceeds and the gated image data 58 may be generated. If however, a scan parameter is not met for a slice, a determination may be made at decision block 80 and the acquisition process stopped 82. The operator may be notified of the acquisition failure and may reinitiate the scan, if desired, at step 84. Alternatively, reinitiation of the scan may be automated such that the operator simply awaits the successful completion of the scan procedure or the failure of the scan procedure based on a timeout or other failure criterion. In this manner, acquisition may be allowed to proceed until the specified image data has been acquired during the desired low-motion intervals. While an MR example was discussed with regard to FIG. 5, one of ordinary skill in the art will readily apprehend that the CT, X-ray and other imaging modality acquisition protocols may take advantage of the present technique in this manner.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

1. A method for gating image data, comprising the steps of:
acquiring a set of motion data during a breath hold;
deriving one or more attributes of motion from the set of motion data;
deriving an initiation threshold and a termination threshold from the one or more attributes; and
generating a set of gated image data using one or more gating intervals derived from the initiation threshold and the termination threshold.

2. The method as recited in claim 1, wherein acquiring the set of motion data comprises acquiring the set of motion data from at least one of a set of pre-acquisition image data, a set of image data, and one or more sets of sensor data.

3. The method as recited in claim 1, wherein acquiring the set of motion data comprises measuring at least one of a displacement, a pressure, an acceleration, a velocity, and a pressure via one or more non-electrical sensors.

4. The method as recited in claims 1, wherein acquiring the set of motion data comprises measuring at least one of an electrical activity indicating a muscular contraction and a change in electrical impedance via two or more electrical sensors.

5. The method as recited in claim 1, wherein generating the set of gated image data comprises acquiring the set of gated image data using an imaging system such that acquisition begins when a first measurement of motion decreases below the initiation threshold and acquisition ceases when a second measurement of motion increase above the termination threshold.

6. The method as recited in claim 1, wherein generating the set of gated image data comprises selecting the set of gated image data from a set of image data such that selection begins when a first measurement of motion decreases below the initiation threshold and selection ceases when a second measurement of motion increase above the termination threshold, wherein the first and second measurement of motion are acquired concurrently with the image data.

7. The method as recited in claim 1, wherein the initiation threshold corresponds to the beginning of the breath-hold and the termination threshold corresponds to the cessation of the breath-hold.

8. The method as recited in claim 1, wherein the initiation threshold corresponds to the beginning of a quiet period within the breath hold and the termination threshold corresponds to the end of the quiet period.

9. The method as recited in claim 1, further comprising the steps of:
displaying at least one of the set of motion data, the one or more attributes, the initiation and termination thresholds, and the one or more suggested gating intervals;
determining if at least one of the initiation and termination thresholds and the one or more suggested gating intervals are acceptable; and
replacing at least one of the initiation and termination thresholds and the one or more suggested gating intervals if they are determined to be unacceptable.

10. The method as recited in claim 1, wherein generating the set of gated image data comprises:
determining if one or more scan parameters are satisfied; and
acquiring the set of gated image data if the one or more scan parameters are satisfied.

11. The method as recited in claim 10, further comprising the step of generating a notification if the one or more scan parameters are not satisfied.

12. The method as recited in claim 1, further comprising the step of providing a notification to at least one of a patient and an operator indicating a breath hold status.

13. A computer program, provided on one or more computer readable media, for gating image data, comprising:
a routine for acquiring a set of motion data during a breath hold;
a routine for deriving one or more attributes of motion from the set of motion data;
a routine for deriving an initiation threshold and a termination threshold from the one or more attributes; and
a routine for generating a set of gated image data using the initiation threshold and the termination threshold

14. The computer program as recited in claim 13, wherein the routine for acquiring acquires the set of motion data from at least one of a set of pre-acquisition image data, a set of image data, and one or more sets of sensor data.

15. The computer program as recited in claim 13, wherein the routine for acquiring measures at least one of a displacement, a pressure, an acceleration, a velocity, and a pressure via one or more non-electrical sensors.

16. The computer program as recited in claim 13, wherein the routine for acquiring measures at least one of an electrical activity indicating a muscular contraction and a change in electrical impedance via two or more electrical sensors.

17. The computer program as recited in claim 13, wherein the routine for generating acquires the set of gated image data using an imaging system such that acquisition begins when a first measurement of motion decreases below the initiation threshold and acquisition ceases when a second measurement of motion increase above the termination threshold.

18. The computer program as recited in claim 13, wherein the routine for generating selects the set of gated image data from a set of image data such that selection begins when a first measurement of motion decreases below the initiation threshold and selection ceases when a second measurement of motion increase above the termination threshold, wherein the first and second measurement of motion are acquired concurrently with the image data.

19. The computer program as recited in claim 13, wherein the initiation threshold corresponds to the beginning of the breath-hold and the termination threshold corresponds to the cessation of the breath-hold.

20. The computer program as recited in claim 13, wherein the initiation threshold corresponds to the beginning of a quiet period within the breath hold and the termination threshold corresponds to the end of the quiet period.

21. The computer program as recited in claim 13, further comprising:
a routine for displaying at least one of the set of motion data, the one or more attributes, the initiation and termination thresholds, and the one or more suggested gating intervals; and
a routine for replacing at least one of the initiation and termination thresholds and the one or more suggested gating intervals if they are determined to be unacceptable.

22. The computer program as recited in claim 13, wherein the routine for generating determines if one or more scan parameters are satisfied and acquires the set of gated image data if the one or more scan parameters are satisfied.

23. The computer program as recited in claim 22, comprising a routine for generating a notification if the one or more scan parameters are not satisfied.

24. The computer program as recited in claim 13, comprising a routine for providing a notification to at least one of a patient and an operator indicating a breath hold status.

25. An imaging system comprising,
an imager configured to generate a plurality of signals representative of one or more structures within a region of interest;
data acquisition circuitry configured to acquire the plurality of signals;
data processing circuitry configured to process the plurality of signals;
system control circuitry configured to operate at least one of the imager and the data acquisition circuitry and to generate a set of gated image data from the plurality of signals using one or more gating intervals, wherein the one or more gating intervals are derived from an initiation threshold and a termination threshold, wherein the initiation threshold and the termination threshold are derived from one or more motion attributes derived from a set of motion data acquired during a breath hold; and
an operator workstation configured to communicate with the system control circuitry and to display one or more images generated from the gated image data.

26. The imaging system as recited in claim 25, further comprising a sensor-based motion determination system configured to acquire the set of motion data.

27. The imaging system as recited in claim 26, wherein the sensor-based motion determination system measures electrical attributes of one or more organs.

28. The imaging system as recited in claim 26, wherein the sensor-based motion determination system measures non-electrical attributes of one or more organs.

29. The imaging system as recited in claim 28, wherein one or more non-electrical sensors of the sensor-based motion determination system comprise accelerometers, optical markers, displacement sensors, force sensors, ultrasonic sensors, strain gauges, photodiodes, and pressure sensors.

30. The imaging system as recited in claim 25, wherein the system control circuitry generates the set of gated image data by activating at least one of the imager and the data acquisition circuitry based upon the one or more gating intervals.

31. The imaging system as recited in claim 25, wherein the system control circuitry generates the set of gated image data by selectively processing the plurality of signals based upon the one or more gating intervals.

32. The imaging system as recited in claim 25, further comprising a feedback device configured to notify at least one of a patient and an operator of a breath hold status of the patient based upon data from at least one of a sensor-based motion determination system, the data processing circuitry, and the system control circuitry.

33. The imaging system as recited in claim 32, wherein the feedback device comprises a visual display device configured to display at least one of one or more colors, one or more symbols, and one or more textual messages.

34. The imaging system as recited in claim 32, wherein the feedback device comprises an audible notification device configured to play at least one of one or more tones and one or more audible messages.

35. An imaging system, comprising:
means for acquiring a set of motion data during a breath hold;
means for deriving one or more attributes of motion from the set of respiratory motion data;
means for deriving an initiation threshold and a termination threshold from the one or more attributes; and
means for generating a set of gated image data using one or more gating intervals derived from the initiation threshold and the termination threshold.
